(19) United States
(12) Patent Application Publication
(10) Pub. No.: US 2008/0223375 A1
(43) Pub. Date: Sep. 18, 2008

(54) SINGLE NASAL PRONG NASAL CANNULA

(75) Inventors: Felino V. Cortez, Bowie, MD (US); Douglas Eric Petsinger, Decatur, GA (US); William F. Niland, Arnold, MD (US); Peter Boyd, Charlottesville, VA (US)

Correspondence Address: RATNERPRESTIA, P O BOX 980, VALLEY FORGE, PA, 19482-0980, US

(73) Assignee: Vapotherm, Inc., Stevensville, MD (US)

(21) Appl. No.: 11/940,793

(22) Filed: Nov. 15, 2007

Related U.S. Application Data
(60) Provisional application No. 60/859,220, filed on Nov. 15, 2006.

(51) Int. Cl. A61M 15/08

(52) U.S. Cl. 128/207.18

(57) ABSTRACT

A device and system for delivering breathing gas to a patient is disclosed. The device includes a single nasal prong insertable into a nare. The single nasal prong is sized to allow gas passage between the single nasal prong and the nare. The device also includes a lumen in fluid communication with the single nasal prong. A method of delivering breathing gas to the patient is also disclosed.

### CROSS-REFERENCE TO RELATED APPLICATION

The present application claims priority from U.S. Provisional Patent Application Ser. No. 60/859,220, filed on Nov. 15, 2006, which is incorporated by reference herein in its entirety.

### FIELD OF THE INVENTION

The present invention relates to a nasal cannula for delivering breathing gas to a single nare of a patient for inhalation of the breathing gas.

### BACKGROUND OF THE INVENTION

A standard nasal cannula is equipped with two prongs that extend into a patient's nares. Gas flow is delivered by connecting the nasal cannula to a source of breathing gas. However, because both of the patient's nostrils can be at least partially obstructed by its two prongs, a standard nasal cannula can inhibit the patient's ability to exhale waste gases.

### SUMMARY OF THE INVENTION

Briefly, the present invention provides a device for delivering breathing gas to a patient. The device comprises a single nasal prong insertable into a nare of the patient. The single nasal prong is sized to allow gas passage between the single nasal prong and the nare. A lumen is in fluid flow communication with the single nasal prong.

Additionally, the present invention provides a system for delivering breathing gas to a single nare of a patient. The system comprises a source of breathing gas and a device for delivering breathing gas to a patient. The device comprises a single nasal prong insertable into a nare of the patient. The single nasal prong is sized to allow gas passage between the single nasal prong and the nare. A lumen is in fluid flow communication with the single nasal prong.

Further, the present invention provides a method of administering breathing gas to a single nare of a patient. The method comprises fitting the single nare with a single nasal prong nasal cannula coupled to a source of the breathing gas such that a space exists between the single nasal prong nasal cannula and the single nare; and delivering the breathing gas to a selected nare of the patient.

Also, the present invention provides a device for delivering breathing is gas to a patient. The device includes a nasal element having a single nasal prong insertable into a nare. The single nasal prong is sized to fit loosely within the nare. The device also includes a lumen in fluid communication with the nasal element and a tubing portion in fluid communication with the lumen. A connector is adapted to receive a breathing gas delivery line. The connector is in fluid communication with the tubing portion.

### BRIEF DESCRIPTION OF THE DRAWINGS

The foregoing summary, as well as the following detailed description of preferred embodiments of the invention, will be better understood when read in conjunction with the appended drawings, which are incorporated herein and constitute part of this specification. For the purposes of illustrating the invention, there are shown in the drawings exemplary embodiments of the invention. It should be understood, however, that the invention is not limited to the precise arrangements and instrumentalities shown. In the drawings, the same reference numerals are employed for designating the same elements throughout the several figures. In the drawings:

FIG. 1 is a front elevational view of an embodiment of a nasal cannula according to a first embodiment of the present invention, with the nasal cannula in use on a patient;

FIG. 2 is a schematic view of a system for delivering breathing gas to a patient, utilizing the nasal cannula of FIG. 1;

FIG. 3 is an enlarged top plan view of the nasal cannula from FIG. 2;

FIG. 4 is a side elevational view of the nasal cannula taken along lines 4-4 of FIG. 3;

FIG. 5 is a sectional view of the nasal cannula taken along lines 5-5 of FIG. 4;

FIG. 6 is a perspective view of a nasal cannula according to an alternative embodiment of the present invention;

FIG. 7 is a perspective view of a nasal cannula according to another embodiment of the present invention;

FIG. 8 is a front elevational view of a nasal prong used in the nasal cannula of FIG. 7;

FIG. 9 is a side elevational view of the nasal prong shown in FIG. 8;

FIG. 10 is a perspective view, partially in section of a coupling between the nasal cannula of FIG. 7 and a delivery tube according to the present invention;

FIG. 11 is a top plan view of a nasal cannula according to another embodiment of the present invention; and

FIG. 12 is an enlarged portion of the nasal cannula of FIG. 11, taken along enlargement line 12 of FIG. 11.

### DETAILED DESCRIPTION OF THE INVENTION

The following describes an exemplary embodiment of the invention. It should be understood based on this disclosure, however, that the invention is not limited by the exemplary embodiment of the invention.

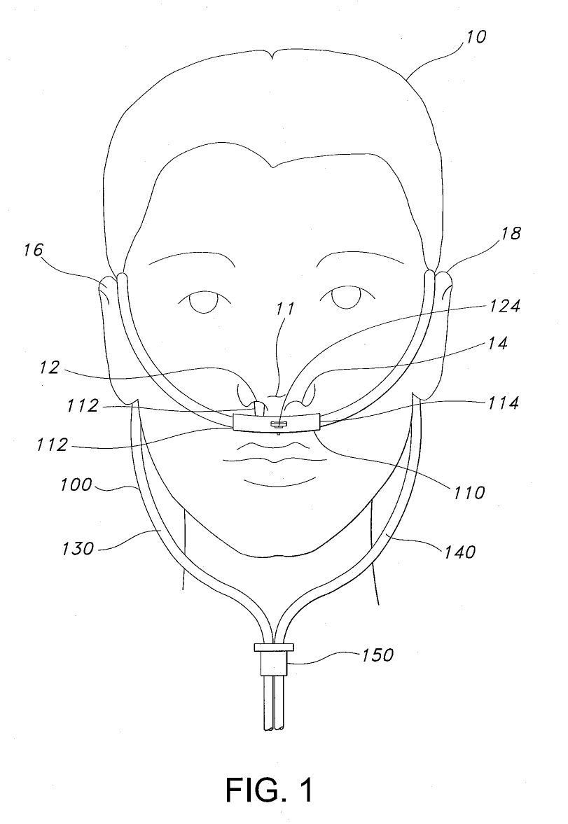

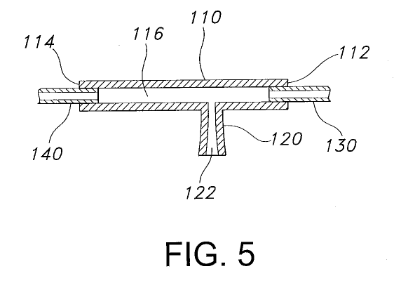

Referring to FIGS. 1-5 generally, an exemplary embodiment of a device in the form of a nasal cannula 100 is provided for delivering breathing gas to a patient. Nasal cannula 100 includes a single nasal prong 120 insertable into a nare 12 of patient 10, wherein single nasal prong 120 may be sized to allow gas passage between single nasal prong 120 and nare 12. Alternatively, single nasal prong 120 may be sized to fully occlude nare 12. Cannula 100 also includes a pair of lumens 130, 140 in fluid flow communication with single nasal prong 120.

A breathing system 60 is also provided for delivering breathing gas to a patient. Breathing system 60 includes a source 170 of breathing gas as well as a breathing gas delivery apparatus such as single nasal prong cannula 100.

In use, breathing gas is administered to a patient by fitting one nare 12 of patient 10 with a single nasal prong 120 of nasal cannula 100 such that a space exists between single nasal prong 120 of nasal cannula 100 and nare 12 of patient 10. The breathing gas is delivered to nare 12 of patient 10 through single nasal prong 120 while evacuating a portion of expired gas through the space between single nasal prong 120 of nasal cannula 100 and nare 12 of patient 10. In between breaths, the breathing gas flushes carbon dioxide from a dead space in the upper respiratory tract via open nare 14.

Open nare 14 may also act as a pressure relief in the event that the flow rate of breathing gas delivered to the patient exceeds the flow rate of breathing gas required by the patient. Excess breathing gas enters the patient's respiratory tract and exits open nare 14, thereby reducing the potential of overpressurizing the patient's lungs.

During exhalation and if the other nare 14 is not obstructed, flow from single nasal prong 120 flows to the user's pharynx, reverses around the end of the septum and flows out through nare 14. During inhalation with the single prong flow rate set higher than maximum inspiration flow rate, excess breathing gas flows out through other nare. An advantage of this approach is that upper airway dead space can be completely flushed of carbon dioxide. This means an increase in PO₂, without supplemental oxygen.

In an exemplary embodiment, depending upon the respiratory therapy being applied, the breathing gas is delivered through single nasal prong 120 of nasal cannula 100 at a rate of between about 1 and 40 liters per minute. In an exemplary embodiment for lower flow applications, the breathing gas is delivered at a rate of between about 1 and 8 liters per minute.

Additionally, the breathing gas is optionally delivered in a humidified condition of up to 100% humidity and at an elevated temperature of between about 33.0° C. to about 43.0° C.

Referring to FIG. 1, an exemplary single nasal prong nasal cannula 100 according to the present invention is shown in use on patient 10. Single nasal prong nasal cannula 100 is a dual hollow lumen tube for delivering breathing gases to patient 10 by single nostril route. Cannula 100 includes a nasal element 110 that is disposed below nose 11 at nares 12 and 14 of patient 10. Nasal element 110 includes a first element end 112 and a second element end 114.

Cannula 100 also includes a first lumen 130 that extends from first element end 112 of nasal element 110, over the right ear 16 of patient 10, and to the front of patient 10. Cannula 100 also includes a second lumen 140 that extends from second element end 114 of nasal element 110, over the left ear 18 of patient 10, and to the front of patient 10, where second lumen 140 is joined next to first lumen 130 at a collar 150, which is slidable up and down first and second lumens 130 and 140.

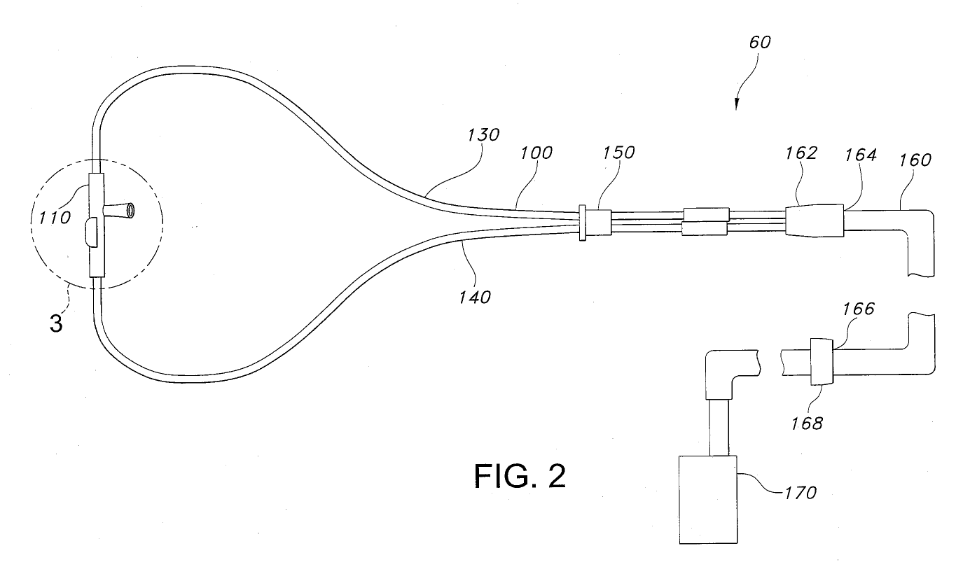

Referring to FIG. 2, in an exemplary embodiment, first and second lumens 130 and 140 are merged into a delivery tube 160 at a hub 162. A first tubing end 164 is fixedly connected to hub 162, while a second tubing end 166 is connected to a tubing connector 168. Tubing connector 168 is connected to a source of breathing gas 170, such as a bottle of compressed oxygen, a vapor-phase humidification system, or any other source of breathing gas. It will be understood by those skilled in the art how to make suitable tubing connections.

Source of breathing gas 170, delivery tube 160, first and second lumens 130 and 140, and nasal element 110 are all in fluid communication, respectively, with each other to form breathing system 60 such that gas from source of breathing gas 170 flows through tubing connector 168, through delivery tube 160, through hub 162, through first and second lumens 130, 140, and to nasal element 110 for breathing by patient 10.

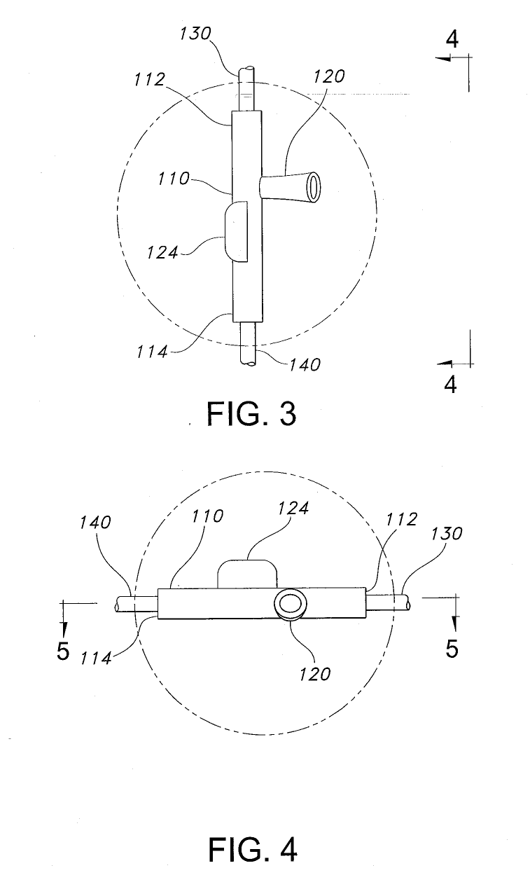

Referring now to FIGS. 3-5, nasal element 110 is shown as a generally elongated tube having first element end 112 and second element end 114, and a nasal element passageway 116 extending therebetween. Single nasal prong 120 extends generally transversely from nasal element passageway 116. As can be seen from FIGS. 3-5, single nasal prong 120 may be asymmetrically disposed on nasal element 110, meaning that single nasal prong 120 is closer to first element end 112 than second element end 114. Single nasal prong 120 defines a nasal passageway 122 that provides fluid communication between nasal element passageway 116 and the atmosphere (or nares when single nasal prong 120 is inserted into the nares). In an exemplary embodiment, single nasal prong 120 and nasal passageway 122 each taper from a smaller diameter to a larger diameter from nasal element passageway 116 to the atmosphere. Such a taper may be referred to as a “reverse taper” because the taper is not used to plug an opening, such as nare 12. Additionally, single nasal prong 120 and nasal passageway 122 each preferably slightly curve in a plane perpendicular to nasal element 110 in a generally arcuate cross sectional shape to facilitate insertion of single nasal prong 120 into nare 12.

Single nasal prong 120 is sized to allow for ease of insertion into nare 12, yet also to provide space between single nasal prong 120 and nare 12 to allow exhaust gas from patient 10 to flow between single nasal prong 120 and nare 12 in order to facilitate exhaust breathing by patient 10. In an exemplary embodiment, single nasal prong 120 fits loosely within nare 12. In this embodiment, single nasal prong 120 is not “wedged” into nare 12 in order to permit flow of exhaust gases past single nasal prong 120 and also to reduce the possibility of inflammation of nare 12 due to excess pressure exerted against nare 12 by single nasal prong 120.

A grasping member 124 extends from nasal element 110 between single nasal prong 120 and second element end 114. Grasping member 124 is spaced approximately ninety degrees around nasal element 110 from single nasal prong 120 to facilitate gripping of grasping member 124 by patient 10 to insert single nasal prong 120 into nare 12 and to remove single nasal prong 120 from nare 12.

In an exemplary embodiment, nasal element 110 and first and second lumens 130 and 140 are constructed from silicone or some other flexible, suitable biocompatible material, as will be understood by those skilled in the art. In an exemplary embodiment, collar 150 is constructed from polyethylene, polypropylene or polyvinyl chloride, or some other suitable polymer.

To insert single nasal prong nasal cannula 100, patient 10 places first lumen 130 over right ear 16 and places second lumen 140 over left ear 18. Patient 10 then places nasal element 110 under nose 11 such that single nasal prong 120 is located just below selected nare 12, 14 for insertion of single nasal prong 120. Patient 10 may use grasping member 124 to manipulate nasal element 110 into position.

Patient 10 inserts single nasal prong 120 into selected nare 12, 14 and releases nasal element 110. As seen in FIG. 1, right nare 12 is selected, by way of example only. Single nasal prong 120 is inserted inside nare 12 without exerting undue pressure on the inside of the nostril. Collar 150 is adjusted along the length of first and second lumens 130, 140 to tighten single nasal prong nasal cannula 100 under the chin of patient 10 to assist in retaining single nasal prong 120 in nare 12.

By adjusting collar 150 under the chin, first and second lumens 130, 140 are drawn relatively tightly against the skin of patient 10, allowing the patient's body heat to help maintain temperature when delivering heated humidified gas. This feature reduces the ambient temperature gradient between the heated and humidified breathing gas and atmosphere, and further leads to reduced condensation in first and second lumens 130, 140, preventing rain-out and liquid droplets delivered into patient's nose 11.

In an exemplary embodiment, a single nasal prong 120 does not totally occlude the nostril passageway in order to allow exhaust gases to pass through the nostril, and between single nasal prong 120 and nare 12. Additionally, excess pressure by single nasal prong 120 against the nasal walls may stimulate the nasal mucosa in that nostril which could increase secretions to rid itself of single nasal prong 120, leading to single nasal prong 120 to possibly “pop out” of the nostril. Further, extreme pressure may collapse the capillaries within the nostril, leading to tissue necrosis.

In operation, since single nasal prong 120 is inserted only into one nare 12, as described above, nasal cannula 100 permits remaining nare 14 to remain open to facilitate further exhalation of waste gases (e.g. carbon dioxide). Single nasal prong 120 can be alternated between nare 12 and nare 14 to reduce skin and nasal mucosal irritation. Alternatively, remaining nare 14 may be used to facilitate insertion of an additional device into patient's nose 11, such as a nasogastric tube, a suction tube, or monitoring equipment (not shown).

A nasal cannula according to aspects of this invention is well suited for delivering breathing gas to a patient under a variety of conditions. For example, nasal cannula 100 is well suited for delivering heated and humidified breathing gas to a patient for respiratory therapy. Such breathing gas can be delivered, for example, via nasal cannula 100 using an apparatus capable of operating in a controlled air output temperature range of from about 33° C. to about 43° C. and an operating flow range of about 1 to about 40 l/min. An example of such an apparatus is described in application Ser. No. 10/149,356, filed Jan. 29, 2003, which is incorporated herein by reference. Also, gas is optionally delivered through nasal cannula 100 is at to a flow rate in a range of about 1 liter per minute to about 8 liters per minute as disclosed in application Ser. No. 10/810,768, filed Mar. 26, 2004, also incorporated herein by reference.

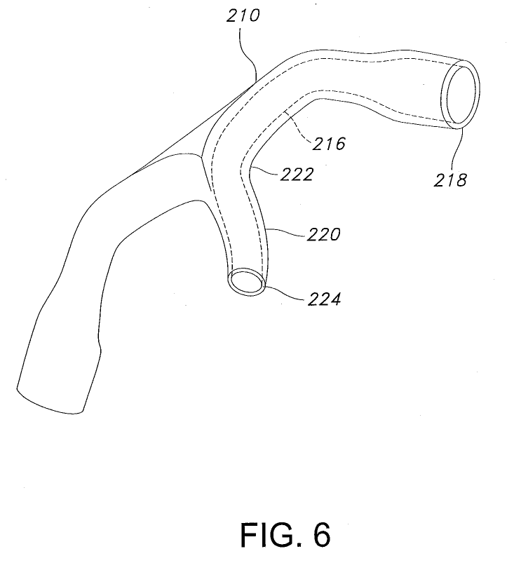

FIG. 6 shows an alternative embodiment of a nasal element 210 that includes a curved lumen 216 extending therethrough from an input end 218 to an output, or distal, end 224. A single nasal prong 220 is symmetrically disposed on nasal element 210. Nasal prong 220 tapers from a larger diameter to a smaller diameter from a transition portion 222 to distal end 224. While nasal element 210 does not include a grasping member, as is provided in nasal element 110 as shown in FIGS. 1-4, those skilled in the art will recognize that a grasping member can be added.

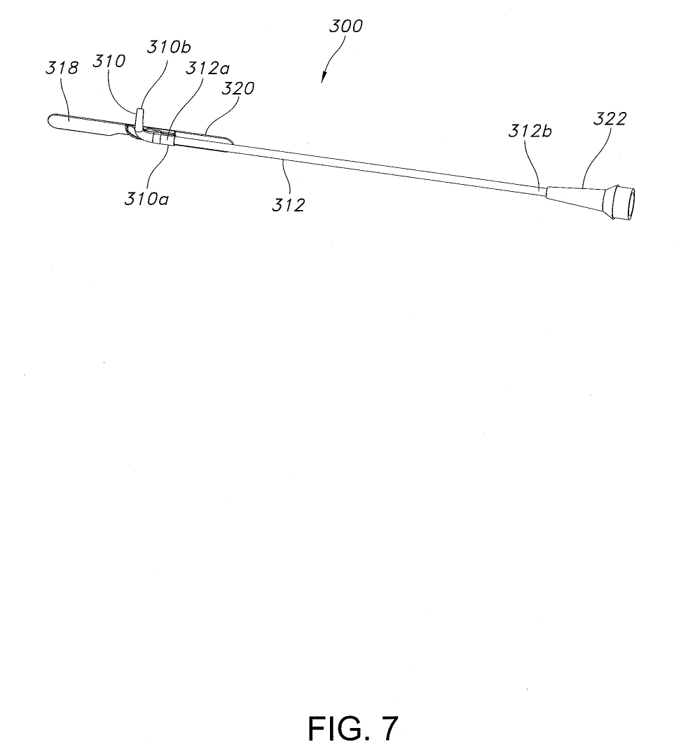

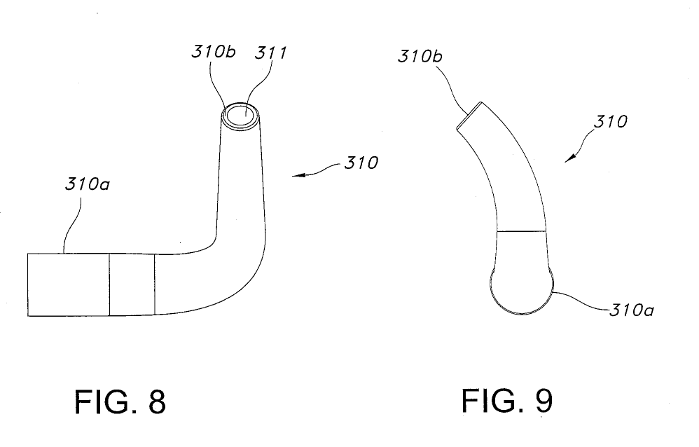

FIG. 7 shows yet another embodiment of a cannula 300 according to the present invention. Cannula 300 includes a single nasal prong 310 extending from a first end 312a of a single lumen 312. Front and side views of single nasal prong 310 are shown in FIGS. 8 and 9. Nasal prong 310 has a coupled end 310a that is fixedly coupled to lumen 312. Nasal prong 310 also includes a free end 310b that is inserted into a user's nare (not shown). Free end 310b extends at an angle, such as about ninety degrees, from coupled end 310a. Single nasal prong 310 changes direction via a smooth curve between coupled end 310a and free end 310b. Inner lumen 311 of single nasal prong 310 tapers from a larger diameter to a smaller diameter from coupled end 310a to free end 310b, with a generally constant wall thickness.

Adhesive strips 318, 320 extend from nasal prong 310. A first adhesive strip 318 extends away from lumen 312 and a second adhesive strip 320 extends along lumen 312. Adhesive strips 318, 320 are used to releasably secure cannula 300 to a user during use. Cannula 310 may be applicable for pediatric and/or neo-natal use, where it may be impractical to attempt to configure lumen 312 over the user's ear.

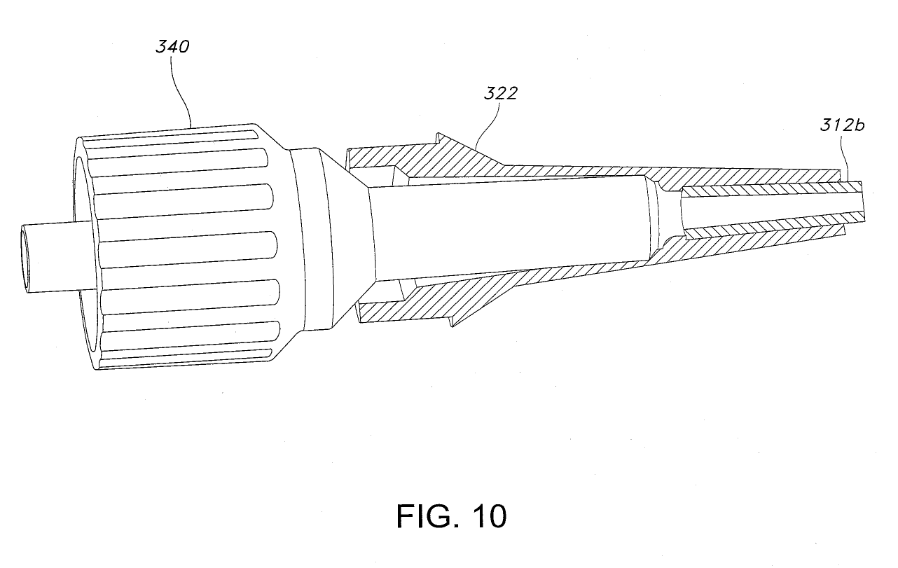

A second end 312b of lumen 312 includes a hub 322 that is releasably connectable to a breathing gas supply, such as delivery tube 160 shown in FIG. 2. A connection between hub 322 and a fitting 340 for the delivery tube is shown in FIG. 10. The inner diameter of lumen 312 is smaller than the inner diameter of delivery tube fitting 340.

Unlike cannula 100 that is draped over the user's ears during use, lo cannula 300 is adhered to the user by adhering adhesive strips 318, 320 to an area between the user's top lip and nose.

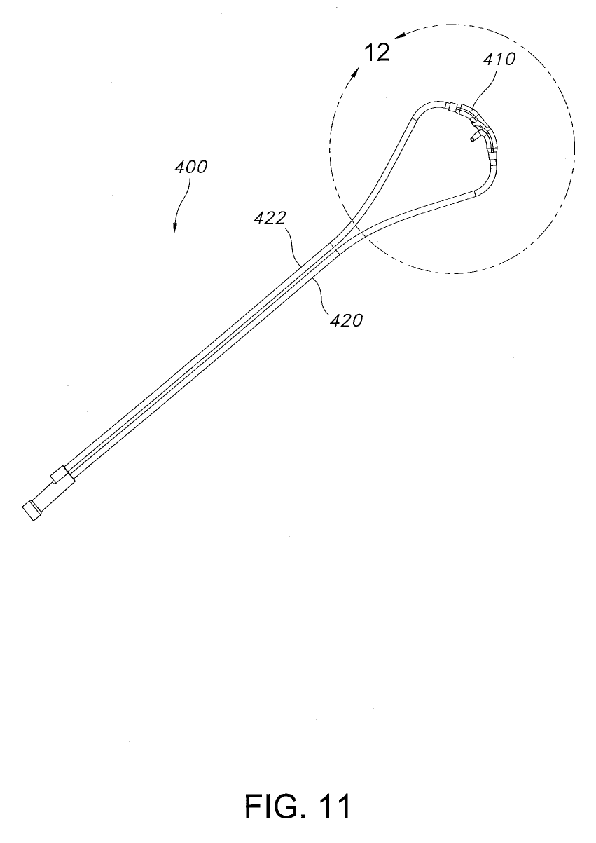

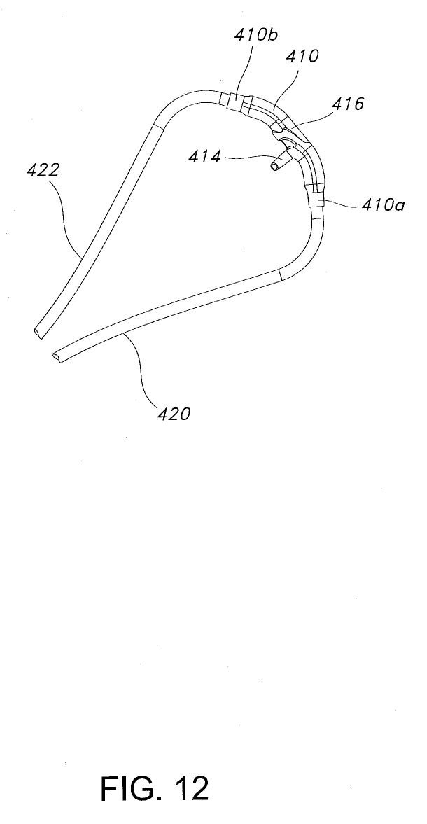

An alternative embodiment of a nasal cannula 400 according to an embodiment of the present invention is shown in FIGS. 11 and 12. Nasal cannula 400 is designed to be used over the user's ears, such as is shown in FIG. 1. A nasal prong 410 includes a first end 410a that is coupled to a supply lumen 420 and a second end 410b that is coupled to a closed lumen 422. Breathing gas enters first end 410a through supply lumen 420. Closed lumen 422 does not permit flow of breathing gas therethrough, but serves as an anchor over the user's ear.

Nasal prong 410 is split into two paths, which include a single nasal lumen 414 and a second path 416, which is not in fluid communication with the user. Single nasal lumen 414 curves about 90 degrees relative to first end 410a of nasal prong 410.

The structures of nasal prongs 210, 310, 410, with their smooth curves, provide for a smooth flow of breathing gas to the user, which minimizes noise of the breathing gas as it flows though the respective nasal prongs 210, 310, 410. Also, the taper of the inner lumens in nasal prongs 210, 310, 410 also eliminates any breathing gas expansion area that may induce rainout. The taper may be a gradual taper as shown in nasal prongs 210, 310, 410, or alternatively, the taper may be in a stepped fashion. The taper is designed to reduce or eliminate flow disruptions that could cause rainout when delivering heated and humidified gas.

The ability to deliver CPAP flow via a single prong at higher flows has enhanced the standard of care for establishing a bridge from endo-tracheal mechanical ventilation. The single prong nasal cannula according to the present invention frees one nare for feeding tubes and nasogastric tubes for a gastric vent. This is extremely helpful with the left sided obstruction, Coarctation of the aorta (COA) who will have a thoracotomy and lung deflation and retraction for surgical access. Post extubation support with proactive use of CPAP via the inventive single prong nasal cannula has decreased reintubation for post-operative failure. This patient population has the possibility of gastric reperfusion injury and the nasal-gastric tube is necessary. Using CPAP with the inventive nasal cannula may also play a role in decreasing reintubation for the post-operative diaphragm lethargy due to phernic nerve inflammation from manipulation.

Also, a nasal cannula according to the present invention, in conjunction with CPAP, may be used with a patient that may present as a difficult challenge from discontinuing from endo-tracheal mechanical ventilation for pure respiratory concerns or hemodynamically driven respiratory failure.

Further, long term use of the inventive nasal cannula may reduce breakdown issues for the face or the nares. Additionally, patient nutritional issues can be effectively addressed by placement of a naso-jejunal gastric tube for continuous feeds while on CPAP using the inventive nasal cannula. Starting flow rate of breathing gas with the inventive nasal cannula for a 2-4 Kg patient is between about 8 and about 10 liters per minute. This flow rate is titrated up or down based on auscultated CPAP sounds, work of breathing (WOB), hemodynamics, and SpO2 of the patient. Flows of twenty liters per minute have been used on 4-6 Kg patients.

### EXAMPLE

A nasal cannula according to the present invention was used for delivering CPAP to a patient with a complete cleft palate. Effective CPAP was delivered to the patient with flows at 8 LPM, using blended oxygen. This patient remained on CPAP with the single cannula for two months with FiO2 ranging from 0.30 to 0.16.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A device for delivering breathing gas to a patient comprising:
a) a single nasal prong insertable into a nare of the patient, the single nasal prong sized to allow gas passage between the single nasal prong and the nare; and
b) a lumen in fluid flow communication with the single nasal prong.

2. The device according to claim 1, further comprising a tubing portion in fluid flow communication with the lumen.

3. The device according to claim 2, further comprising a connector adapted to receive the breathing gas, wherein the connector is in fluid flow communication with the tubing portion.

4. The device according to claim 1, wherein the single nasal prong extends from a nasal element.

5. The device according to claim 4, wherein the nasal element comprises a grasping member extending therefrom and positioned to be grasped by a user of the device.

6. The device according to claim 4, wherein the single nasal prong is asymmetrically disposed on the nasal element.

7. The device according to claim 4, further comprising a second lumen in fluid flow communication with the nasal element.

8. The device according to claim 1, wherein the single nasal prong comprises a reverse taper shape.

9. The device according to claim 1, wherein the single nasal prong comprises a generally arcuate cross sectional shape.

10. A system for delivering breathing gas to a patient, the system comprising:
a) a source of breathing gas; and
b) a device for delivering breathing gas to a patient comprising:
i) a single nasal prong insertable into a nare of the patient, the single nasal prong sized to allow gas passage between the single nasal prong and the nare; and
ii) a lumen in fluid flow communication with the single nasal prong.

11. The system according to claim 10, wherein the source of breathing gas comprises a vapor-phase humidification system.

12. A method of administering breathing gas to a patient comprising the steps of:
a) fitting one nare of the patient with a single nasal prong of a nasal cannula such that a space exists between the single nasal prong of the nasal cannula and the nare of the patient; and
b) delivering the breathing gas to the nare of the patient through the single nasal prong while evacuating a portion of gas through the space between the single nasal prong of the nasal cannula and the nare of the patient and through the other nare of the patient.

13. The method according to claim 12, wherein delivering the breathing gas comprises the step of delivering the breathing gas at a rate of between about 5 and 40 liters per minute.

14. The method according to claim 12, wherein delivering the breathing gas comprises the step of delivering the breathing gas at a rate of between about 1 and 8 liters per minute.

15. The method according to claim 12, wherein delivering the breathing gas comprises the step of delivering the breathing gas at a temperature of between about 33.0° C. to about 43.0° C.

16. The method according to claim 12, further comprising the step of inserting a medical device into the other nare and performing a medical procedure on the patient with the medical device.

17. A method of flushing carbon dioxide from an upper airway of a patient comprising the steps of:
inserting a single prong of a single prong nasal cannula into one nare of the patient;
providing breathing gas to the one nare through the single prong;
inhaling the breathing gas through the one nare and into the upper respiratory tract; and
exhaling a portion of the breathing gas through a space between the single prong and the one nare and another portion of the breathing gas through a remaining nare and exhaling the carbon dioxide with the exhaled breathing gas.

18. A method of relieving breathing gas pressure to a patient generated by excess breathing gas flow to the patient comprising the steps of:
inserting a single prong of a single prong nasal cannula into one nare of the patient;
providing a flow rate breathing gas to the one nare through the single prong;
inhaling a portion of the breathing gas through the one nare and into the upper respiratory tract; and
discharging a remaining portion of the breathing gas through a space between the single prong and the one nare and another of the remaining portion of the breathing gas through a remaining nare.
